(19) United States
(12) Patent Application Publication
(10) Pub. No.: US 2018/0147127 A1
(43) Pub. Date: May 31, 2018

(54) COSMETIC COMPOSITION

(71) Applicant: OXITENO S.A. INDÚSTRIA E COMÉRCIO, São Paulo (BR)

(72) Inventors: Alexandra P. Pavanelli Sartori, São Paulo (BR); Elvira Aparecida S. Bizarro Dos Santos, São Paulo (BR); Maria Goretti Delfino Pedreca, São Paulo (BR)

(21) Appl. No.: 15/537,824

(22) PCT Filed: Dec. 18, 2015
(86) PCT No.: PCT/BR2015/000194

§ 371 Date: Oct. 23, 2017

(30) Foreign Application Priority Data
Dec. 19, 2014 (BR) 1020140321330

(51) Int. Cl. A61K 8/44; A61Q 5/12; A61K 8/46; A61Q 5/02

(52) U.S. Cl. CPC A61K 8/44; A61Q 5/02; A61K 8/463; A61Q 5/12

(57) ABSTRACT

The present invention relates to a cosmetic composition comprising between 20 and 45% of a first surfactant, from 0.1 and 22% of a second surfactant; and cosmetically acceptable adjuvants for use in hair cleaning and color maintenance of dyed hair, which can be used as a shampoo and other hair formulations.

### FIELD OF THE INVENTION

This invention belongs to the field of use of cosmetics or personal hygiene preparations; specifically, to the use of cosmetics or personal hygiene preparations for hair treatment.

### STATE OF THE ART

Hair care is increasingly drawing consumers attention, especially in Brazil, which occupies the first place in terms conditioner consumption and the second place in shampoo consumption, surpassing the consumption per capita of most of developed countries. Is an evolving market and already a trendsetter for other regions of the world. Benefits such as color maintenance, softness, smoothness and low irritability are essential requisites that the hair products must deliver to the consumer. For formulators, the challenge is to obtain formulations that meet the expectation of increasingly demanding consumers, combining viable costs and easiness in the production process.

A typical shampoo formulation consists of cleaning agents, foam stabilizers, hair conditioners, thickeners, opacifiers, pearlizing agents, preservatives, fragrances, colorants and additives. Surfactants contribute to achieve most of these properties in the final product, such as appearance, foaming, low irritation and hair cleaning and conditioning.

Surfactants are amphiphilic molecules, i.e. they have polar and non-polar parts, which enable them of working on surfaces and interfaces. Thus, these molecules are of great importance in systems involving water/air, water/oil or water/solids interactions such as, for example, in oil removal from hair and skin. These molecules in solution have the ability to auto associate, forming structured systems, such as micelles spherical, hexagonal or lamellae.

The magnitude of these effects is also related with the balance of solubility between the polar and apolar parts of these molecules, which is named hydrophilic-lipophilic balance (HLB), a factor determinant of physicochemical properties, such as reduction of surface tension, critical micelle concentration (cmc), detergency, foaming and viscosity.

The use of a single surfactant generally does not provide all intended features to a shampoo formulation, being common the use of mixtures. Of these, the most used is the combination of sodium lauryl ether sulfate (SLES), which acts as a detergent base due to its cleaning power, water solubility and high foaming power combined with the use of a co-tensoative, which provides stability of foam and hair thickening, conditioning and softness, among other attributes. However, other combinations of surfactants can bring important and desired attributes to consumers, such as color maintenance, reduction in eye irritability and best conditioning effect to the hair.

Currently, companies working in product development for hair maintenance come with specific ingredients, such as active principles and filters. The patent JP2004292387A describes a shampoo composition having a good foaming and softness power to hair. The patent document EP2198837A1 is a detergent cosmetic composition (I) comprises: (a) one or more anionic agents; (b) one or more carboxylic anionic surfactants different from the anionic surfactants; (c) one or more amphoteric and/or zwitterionic surfactants; (d) one or more glycoside surfactants; (e) one or more cationic polymers; (f) one or more zinc salts; and (g) optionally, one or more UV filters.

The Japanese patent document JP08291028, by its turn, relates to a cosmetic product for hair washing that has preventive effect on dyed hair, containing polyphenols, an anionic surfactant and/or an amphoteric surfactant, and a long-chain alcohol.

Thus, as it can be noted, the state of the art does not mention the compositions containing a mixture of two surfactants for maintaining the color of dyed hair into staples.

### Objective of the Invention

The object of this invention is providing a cosmetic composition that can be used on dyed hair hygiene and color maintenance.

### SUMMARY OF THE INVENTION

The instant invention relates to a cosmetic composition comprising a surfactant, a second surfactant and adjuvants for use in dyed hair hygiene and color maintenance of the color of dyed hair strands.

### BRIEF DESCRIPTION OF THE DRAWINGS

In order to obtain a full and complete view of the object of the invention in question, the object of the patent application has drawings, which are referenced as follows:

FIG. 1 is a graph of color loss measurement by using the composition A compared to the control composition.

FIG. 2 is a graph of color loss measurement by using the composition B compared to the control composition.

FIG. 3 is a graph of color loss measurement by using the composition C compared to the control composition.

FIG. 4 presents a washing hair test: 4A—composition A; 4B—control.

FIG. 5 presents a foaming test: 5A—composition A; 5B—control.

FIG. 6 presents a washing hair test: 6A—composition B; 6B—control.

FIG. 7 presents a foaming test: 7A—composition B; 7B—control.

FIG. 8 presents a washing hair test: 8A—composition C; 8B—control.

FIG. 9 presents a foaming test: 9A—composition C; 9B—control.

FIG. 10 is the graph showing the potential cytotoxic test of composition A compared to the control composition.

FIG. 11 is the graph showing the potential cytotoxic test of composition B compared to the control composition.

FIG. 12 is the graph showing the potential cytotoxic test of composition C compared to the control composition.

### DETAILED DESCRIPTION OF THE INVENTION

This invention refers to a cosmetic composition comprising between 20 and 45% of a first surfactant, between 0.1 and 22% of a second surfactant; and cosmetically acceptable adjuvants for use on dyed hair hygiene and color maintenance of dyed hair strands.

For cosmetic composition purposes of this invention, the quantitative limits of its components are expressed as percentage by weight of the component in the final composition (% w).

The surfactants of this invention are sodium lauryl ether sulfate (SLES), sodium lauryl ether sulfosuccinate, cocamidopropyl betaine, lauryldimethylamine oxide, or a mixture thereof.

Preferably, the first surfactant is sodium lauryl ether sulfate (SLES) and the second surfactant belongs to the group consisting of: sodium lauryl ether sulfosuccinate, cocamidopropyl betaine, lauryldimethylamine oxide or a mixture thereof.

The cosmetically acceptable adjuvants are diluents, saponificant, foaming agents, emollients, rheology modifiers, pH regulators, scavengers, fragrance and others. One person skilled in the art would find obvious adding adjuvants in a minimum or enough amount to make up the cosmetic composition, without interfering, improving, withdrawing or reducing the effect of color maintenance of the dyed hair strands.

Among the adjuvants that may be used in the present invention, sorbeth-450 tristearate (mixture of triester of stearic acid and polyethylene glycol ester of sorbitol in 450 moles of ethylene oxide), water, PEG-cocoate, PEG-32 distearate and PEG-175 distearate may be used in the present invention, in a non-limiting manner.

Preferably, the cosmetic composition comprises between 25 and 40% of a first surfactant, between 1.0 and 20% of a second surfactant; and cosmetically acceptable adjuvants known to a person skilled in the art.

More preferably, the cosmetic composition of the invention comprises between 26 and 38% of a first surfactant, between 1.2 and 18% of a second surfactant; and cosmetically acceptable adjuvants known to a person skilled in the art.

The present invention further relates to the use of the cosmetic composition for dyed hair hygiene and color maintenance, as a shampoo. The shampoos of the present invention are for household and professional use, which can be used in daily or phased washing schemes.

Table 1 below indicates possible modes of the composition, that were obtained within the quantitative limits set out above.

| COMPOSITION | 1st SURFACTANT | 2nd SURFACTANT |
|---|---|---|
| A | Sodium lauryl | Sodium lauryl ether |
| | ether sulphate | sulfosuccinate |
| B | Sodium lauryl | Cocamidopropyl |
| | ether sulphate | Betaine |
| C | Sodium lauryl | Lauryldimethylamine |
| | ether sulphate | oxide |

All compositions additionally have, as ingredients: rheology and pH modifiers, preservatives, scavengers and fragrance.

All compositions listed in table 1 are effective in promoting hair and scalp cleaning, and are able to keep the coloring of dyed hair even after a large number of washes, such as, for example, 30 washes, when compared with a washing control composition.

Although the invention has been widely described, one person skilled in the art would find obvious that many changes and modifications may be made without covering said modifications by the scope of the invention.

The examples below are merely illustrative of the embodiments of the invention, and should not be used to limit the holder rights, who should stick only to the attached set of claims.

### EXAMPLES

### Example 1—Statistical Analysis

Exploratory data analysis was carried out (abstract graphic and measures). The times, for each treatment, were compared to T0 through ANOVA followed by a Dunnett's test. The treatments were compared using the Student t test for independent samples with 95% confidence level. For statistical analysis, we used MINITAB 14 and XLSTAT 2013 software.

The analyses of statistical data were employed for preparing the charts described in the examples below.

### Example 2—Tested Compositions

The following shampoo compositions were produced and are herein described both qualitatively and quantitatively, according to the description given above.

| | Concentration |
|---|---|
| COMPOSITION | (% by weight) |
| A - Sodium lauryl ether | 27 |
| sulfate | |
| Sodium lauryl ether | 17 |
| sulfosuccinate | |
| B - Sodium lauryl ether | 29 |
| sulfate | |
| Cocamidopropyl Betaine | 12 |
| C - Sodium lauryl ether | 35 |
| sulfate | |
| Lauryldimethylamine oxide | 1.5 |

ADDITIONAL COMPONENT IN ALL COMPOSITIONS ABOVE

| | |
|---|---|
| Sorbeth-450 tristearate + | 1 |
| water + PEG-9 cocoate + | |
| PEG-32 distearate + PEG- | |
| 175 distearate. | |
| Water | q.s.p. |
| Other ingredients ¹ | q.s.p. |

Note
¹ For all formulas, the other ingredients are: rheology and pH modifiers, preservatives, scavengers and fragrance.

### Example 3—Evaluation of Loss of Hair Color

The evaluation of color loss in staples was carried out throughout 30 washes, reported as Δ(%) in relation to the initial color. The smaller the Δ value, the greater is the color maintenance.

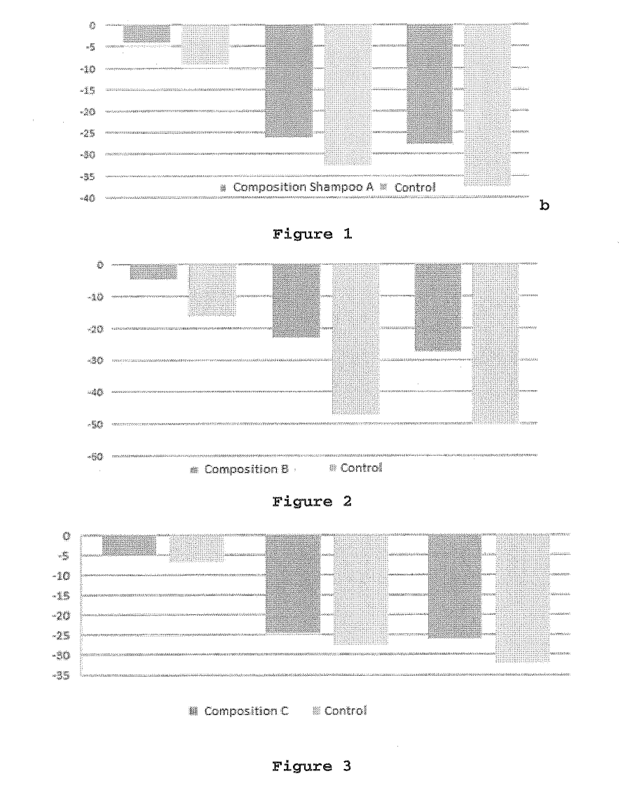

The graph of FIGS. 1, 2 and 3 show the color maintenance of the dyed hair even after 30 washes comparing, respectively, the compositions A, B and C in the control formula with Δ(%) in relation to T0 by treatment.

The data of these figures show that shampoos made with different compositions of the present invention protect more coloring of strands, from the first to last wash, when compared with the control compositions.

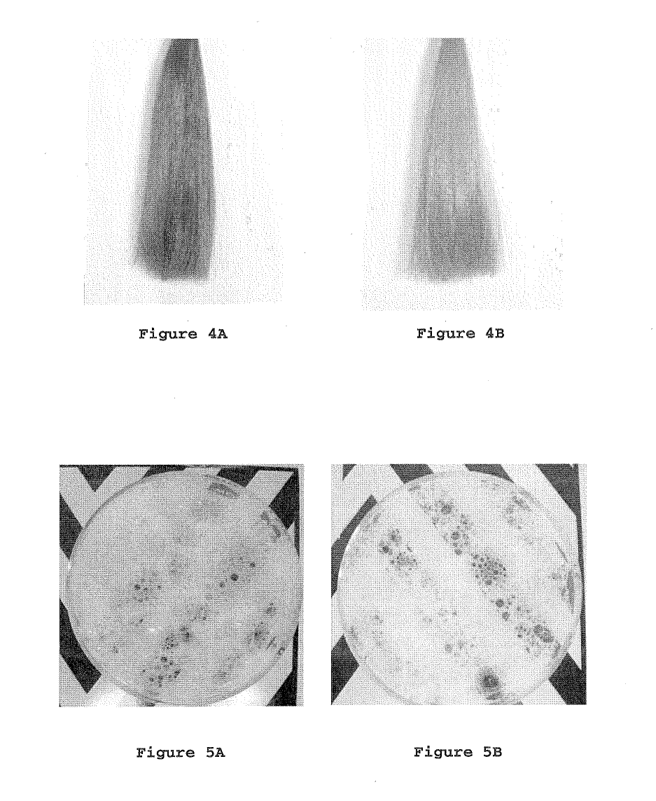

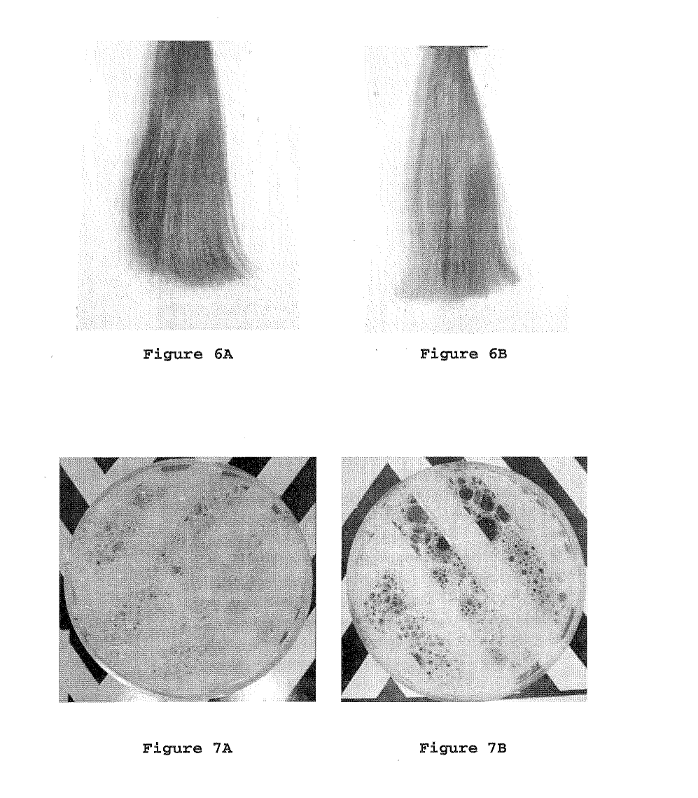

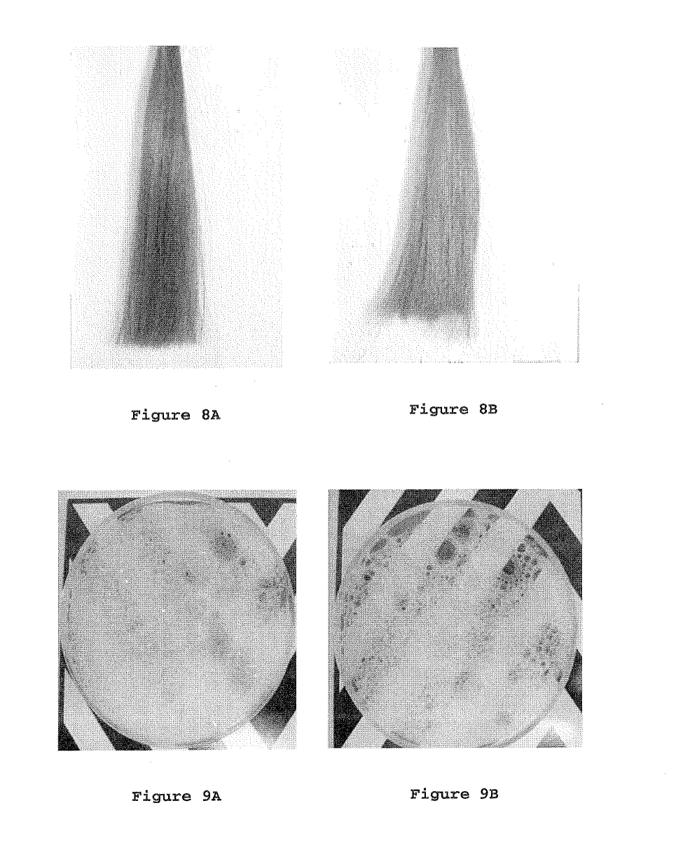

It can be seen in FIG. 4 that the staples, after 30 washes, has a color better and longer, even as a function of time of use of the shampoo composition A, which does not occur with the hair washed with control shampoo. The same procedure was performed with the shampoos of composition B (FIG. 6) and composition C (FIG. 8), giving a result identical to that achieved by composition A.

It can be further noticed, in FIGS. 5, 7 and 9, that the shampoos of the invention generate a creamier and abundant foam when compared to the control. This attribute is fairly valued and is associated with well-being feeling. The sensory provided by rich foam refers to a perception of a better performance by the consumer.

### Example 4—In Vitro Irritation Potential

Evaluation of cytotoxic potential by Balb/C 3T3 NRU method, according to ISO 10993-5 and INVITTOX No. 46 protocol.

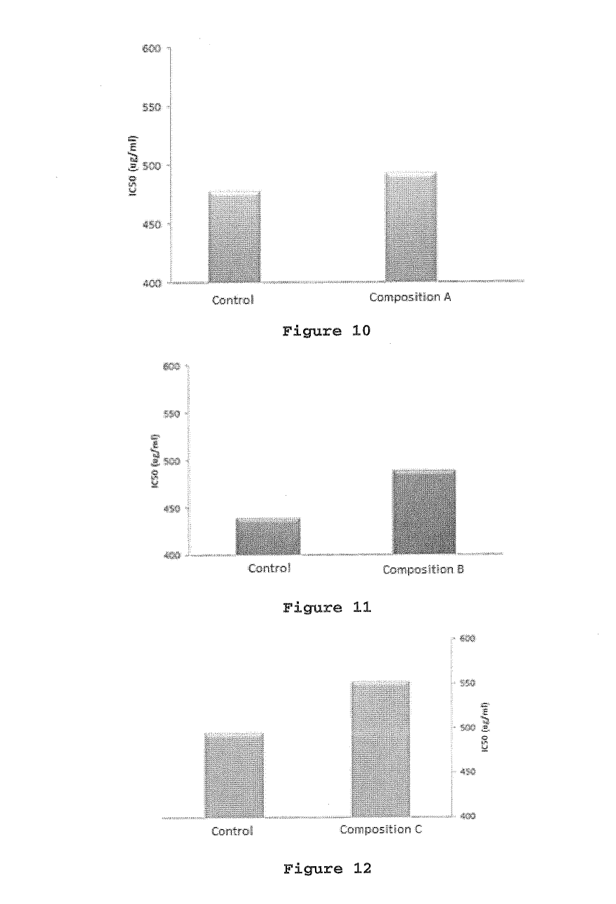

An in vitro comparative study of the irritation potential of the shampoos of this invention are presented, respectively, in FIGS. 10, 11 and 12, and the results show that these compositions are less cytotoxic and softer than the control. The assay showed a deviation between the replicas of nearly 10%.

1. A cosmetic composition comprising between 20 and 45% of a first surfactant, between 0.1 and 22% of a second surfactant; and at least one cosmetically acceptable adjuvant.

2. The composition according to claim 1, wherein the surfactant is selected from the group consisting of sodium lauryl ether sulfate (SLES), sodium lauryl ether sulfosuccinate, cocamidopropyl betaine, lauryldimethylamine oxide or a mixture thereof.

3. The composition according to claim 1, wherein the first surfactant is sodium lauryl ether sulfate (SLES) and the second surfactant is selected from the group consisting of: sodium lauryl ether sulfosuccinate, cocamidopropyl betaine, lauryldimethylamine oxide or a mixture thereof.

4. The composition according to claim 1, wherein the cosmetically acceptable adjuvant is selected from the group consisting of a diluents, a saponificant, a foaming agent, a rheology modifier, a pH regulator, a preservative, a scavenger, a fragrance and a mixture thereof.

5. The composition according to claim 1, wherein the composition comprises between 25 and 40% of the first surfactant, between 1.0 and 20% of the second surfactant; and the cosmetically acceptable adjuvant.

6. The composition according to claim 5, wherein the composition comprises between 26 and 38% of the first surfactant, between 1.2 and 18% of the second surfactant; and the cosmetically acceptable adjuvant.

7. The composition according to claim 2, wherein the first surfactant is sodium lauryl ether sulfate (SLES) and the second surfactant is selected from the group consisting of: sodium lauryl ether sulfosuccinate, cocamidopropyl betaine, lauryldimethylamine oxide or a mixture thereof.

8. The composition according to claim 2, wherein the composition comprises between 25 and 40% of the first surfactant, between 1.0 and 20% of the second surfactant; and the cosmetically acceptable adjuvant.

9. The composition according to claim 3, wherein the composition comprises between 25 and 40% of the first surfactant, between 1.0 and 20% of the second surfactant; and the cosmetically acceptable adjuvant.

10. The composition according to claim 8, wherein the composition comprises between 26 and 38% of the first surfactant, between 1.2 and 18% of the second surfactant; and the cosmetically acceptable adjuvant.

11. The composition according to claim 9, wherein the composition comprises between 26 and 38% of the first surfactant, between 1.2 and 18% of the second surfactant; and the cosmetically acceptable adjuvant.
